(19) United States
(12) Patent Application Publication
(10) Pub. No.: US 2026/0083986 A1
(43) Pub. Date: Mar. 26, 2026

(54) RADIOTHERAPY APPARATUS

(71) Applicant: Hitachi High-Tech Corporation, Tokyo (JP)

(72) Inventor: Tomokazu SHIMAKURA, Tokyo (JP)

(21) Appl. No.: 19/113,451

(22) PCT Filed: Aug. 7, 2023
(86) PCT No.: PCT/JP2023/028769

§ 371 Date: Mar. 20, 2025

(30) Foreign Application Priority Data
Dec. 26, 2022 (JP) 2022-208331

(51) Int. Cl. A61N 5/10

(52) U.S. Cl. CPC A61N 5/1069; A61N 5/1077; A61N 2005/1059; A61N 2005/1087

(57) ABSTRACT

Provided is a radiotherapy apparatus that can suppress an influence of radiation on an imaging apparatus. This X-ray imaging apparatus includes: an Xray tube 7 that irradiates a patient 6 with an X-ray as light for imaging; and an FPD 2 that detects the X-ray through the patient 6. The Xray tube 7 and the FPD 2 are disposed at positions offset from a line connecting the irradiation nozzle 1 and the isocenter 8 of radiation from the irradiation nozzle 1. The FPD 2 is disposed on a reference plane S which is a plane passing through the isocenter 8 and orthogonal to the line connecting the irradiation nozzle 1 and the isocenter 8, or on a side closer to the irradiation nozzle than the reference plane S.

### TECHNICAL FIELD

The present disclosure relates to a radiotherapy apparatus.

### BACKGROUND ART

PTL 1 discloses a particle therapy apparatus capable of irradiating a patient with particle beams from three directions as a radiotherapy apparatus that performs cancer therapy by irradiating a cancer lesion with radiation. The particle therapy apparatus includes an irradiation port through which a particle beam passes, an irradiation nozzle that irradiates a patient with the particle beam from the irradiation port, and a movement mechanism that moves the irradiation nozzle. In addition, three irradiation ports are present, and the three irradiation ports are disposed along the vertical direction, the horizontal direction, and the oblique direction, respectively. The particle therapy apparatus uses the movement mechanism to move the irradiation nozzle so as to be connected to the irradiation port through which the particle beam is guided while switching the irradiation port through which the particle beam is guided. As a result, the patient can be irradiated with radiation from three directions.

### CITATION LIST

### Patent Literature

PTL 1: JP 2017-153908 A

### SUMMARY OF INVENTION

### Technical Problem

The radiotherapy apparatus is provided with, for example, an imaging apparatus that acquires an image of a patient in order to determine the position of the patient, in addition to an irradiation nozzle that emits radiation. For example, the particle therapy apparatus described in PTL 1 includes, as an imaging apparatus, an X-ray generator and a flat panel detector (FPD) that detects an X-ray from the X-ray generator through a patient.

When the imaging apparatus and the irradiation nozzle are disposed independently of each other, the radiation emitted from the irradiation nozzle and the X-ray emitted from the imaging apparatus interfere, and as a result, the imaging apparatus may be affected. For example, in a case where an image of a patient needs to be acquired while radiation is emitted, such as a tumor tracking irradiation process that accurately irradiates organs moving in response to the patient's respiration or the like, the interference of the radiation with the X-ray affects the image of the patient generated by the X-ray, and the accuracy of the image may be reduced.

However, PTL 1 does not describe a positional relationship between the X-ray imaging apparatus and the irradiation nozzle, and it is not possible to suppress the influence of radiation on the imaging apparatus.

An object of the present disclosure is to provide a radiotherapy apparatus that can suppress an influence of radiation on an imaging apparatus.

### Solution to Problem

A radiotherapy apparatus according to one aspect of the present disclosure includes: an irradiation unit which irradiates an irradiation object with radiation; and an imaging apparatus which takes an image of the irradiation object, the imaging apparatus including a light source unit that irradiates the irradiation object with light for imaging; and a detection unit that detects the light through the irradiation object, in which the light source unit and the detection unit are disposed at positions offset from a line connecting an isocenter of the radiation and the irradiation unit, and the detection unit is disposed on a reference plane which is a plane passing through the isocenter and orthogonal to the line connecting the isocenter and the irradiation unit or on a side closer to the irradiation unit than the reference plane.

### Advantageous Effects of Invention

According to the present invention, it is possible to suppress an influence of radiation on an imaging apparatus.

### BRIEF DESCRIPTION OF DRAWINGS

FIG. 1 is a view schematically illustrating an appearance of an irradiation system according to a first embodiment of the present disclosure.

FIG. 2 is a view illustrating an example of a configuration of a radiotherapy apparatus according to the first embodiment of the present disclosure.

FIG. 3 is views illustrating a disposition example of an X-ray imaging apparatus according to the first embodiment of the present disclosure.

FIG. 4 is views illustrating another disposition example of the X-ray imaging apparatus according to the first embodiment of the present disclosure.

FIG. 5 is views illustrating an FPD installable angle range according to the first embodiment of the present disclosure.

FIG. 6 is views illustrating an FPD installable angle range according to a second embodiment of the present disclosure.

FIG. 7 is views illustrating a disposition example of an X-ray imaging apparatus according to a third embodiment of the present disclosure.

FIG. 8 is views illustrating an FPD installable angle range according to a fourth embodiment of the present disclosure.

### DESCRIPTION OF EMBODIMENTS

Hereinafter, embodiments of the present disclosure will be described with reference to the drawings.

### First Embodiment

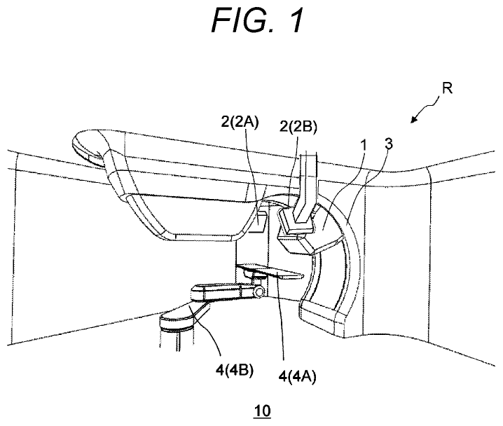

FIG. 1 is a view schematically illustrating the appearance of the irradiation system included in the radiotherapy apparatus according to the first embodiment of the present disclosure. An irradiation system 10 illustrated in FIG. 1 is disposed in a treatment room R where radiotherapy is performed, and includes an irradiation nozzle 1, an FPD2, a nozzle rotating system 3, and a patient support 4.

The irradiation nozzle 1 is an irradiation unit that irradiates a patient: an irradiation object placed on the patient support 4 (not illustrated in FIG. 1) with radiation. The type of radiation is not particularly limited, but in the present embodiment, the radiation is a particle beam, more specifically, a carbon ion beam.

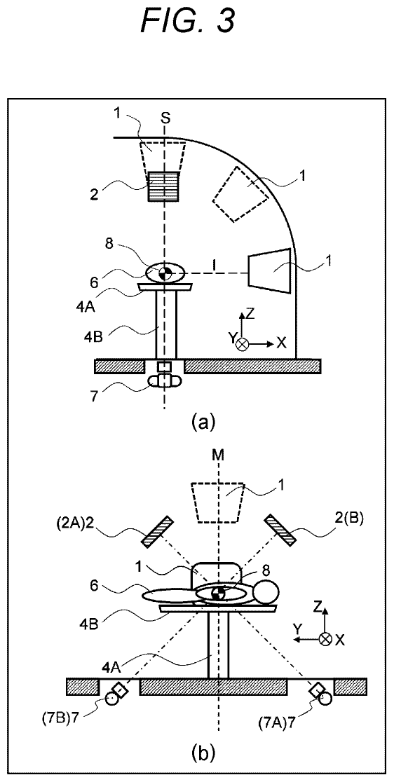

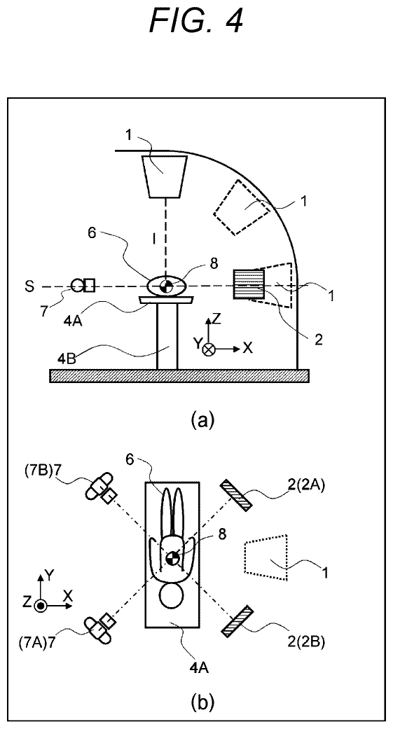

The FPD2 is a part of the imaging apparatus that images a patient and acquires an image of the patient. In the present embodiment, the imaging apparatus is an X-ray imaging apparatus that irradiates a patient with an X-ray as light for imaging and detects the X-ray through the patient to image the patient. The FPD2 is a detection unit (plane detector) that detects an X-ray from an Xray tube (see the Xray tube 7 in FIGS. 3 and 4) functioning as an X-ray light source, which is not illustrated in FIG. 1, through a patient. Although the number of FPDs 2 is not limited, in the present embodiment, two FPDs 2A and 2B are provided as the FPDs 2.

The nozzle rotating system 3 is a movement mechanism that moves the irradiation nozzle 1 and installs the irradiation nozzle 1 at any one of a plurality of irradiation start positions at which radiation is emitted.

The patient support 4 includes a table top 4A as a bed on which a patient is placed and an arm part 4B which moves the table top 4A. The arm part 4B can perform translational movement along each of three axial directions different from one another and rotational movement with each of the three axial directions as a rotation axis with respect to the table top 4A. For example, the arm part 4B is controlled based on the patient image acquired by the X-ray imaging apparatus such that the target volume of the patient placed on the table top 4A coincides with the isocenter of the radiation from the irradiation nozzle 1. The isocenter is a position where radiation is most intensively emitted.

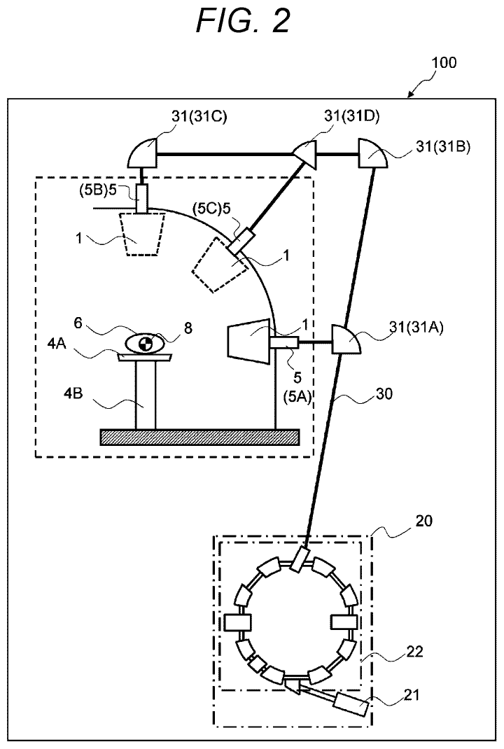

FIG. 2 is a view illustrating an example of the configuration of the radiotherapy apparatus. A radiotherapy apparatus 100 illustrated in FIG. 2 includes an irradiation system 10, an accelerator 20, and a beam transport system 30.

The accelerator 20 accelerates charged particles and extracts the accelerated charged particles as a particle beam. The type of the accelerator 20 is not particularly limited, but in the present embodiment, the accelerator 20 includes a linear accelerator 21 and a synchrotron 22. The linear accelerator 21 is an injection unit that accelerates charged particles and injects the charged particles into the synchrotron 22. The synchrotron 22 further accelerates the charged particles injected from the linear accelerator 21 and extracts the accelerated charged particles as a particle beam (charged particle beam).

The beam transport system 30 is a transfer that transports the particle beam extracted from the accelerator 20 to the irradiation system 10. The beam transport system 30 includes a plurality of deflecting electromagnets 31 that deflects the particle beam to change the traveling direction of the particle beam. In the example of FIG. 2, the beam transport system 30 is provided with four bending magnets 31 A to 31D as the deflecting electromagnets 31.

The irradiation system 10 includes a plurality of irradiation ports 5 in addition to the configuration illustrated in FIG. 1. The irradiation port 5 is a vacuum duct for guiding the particle beam transported by the beam transport system 30 to the irradiation nozzle 1. In the example of FIG. 2, three irradiation ports 5A to 5C are provided as the irradiation ports 5. The irradiation port 5A is provided along a substantially horizontal direction, the irradiation port 5B is provided along a substantially vertical direction, and the irradiation port 5C is provided along an oblique direction (specifically, a direction at an angle of approximately 45 degrees with respect to the horizontal direction).

The irradiation ports 5A to 5C are connected to the beam transport system 30, and the deflecting electromagnets 31 of the beam transport system 30 are controlled to guide the particle beam to any one of the irradiation ports 5A to 5C.

The nozzle rotating system 3 illustrated in FIG. 1 moves the irradiation nozzle 1 and connects the irradiation nozzle 1 to one of the three irradiation ports 5A to 5C. A position connected to each of the irradiation ports 5A to 5C is an irradiation start position. In the present embodiment, the nozzle rotating system 3 rotationally moves the irradiation nozzle 1 about a predetermined rotation axis such that the irradiation nozzle 1 rotates around the patient 6 placed on the patient support 4. The predetermined rotation axis is an axis passing through the isocenter 8 of the radiation emitted from the irradiation nozzle 1, and is oriented in an approximately horizontal direction in the present embodiment. In the present embodiment, the nozzle rotating system 3 rotates the irradiation nozzle 1 by 90° from the horizontal direction to the vertical direction so as to connect the irradiation nozzle 1 to any one of the three irradiation ports 5A to 5C.

with the above configuration, the radiotherapy apparatus 100 uses the nozzle rotating system 3 to move the irradiation nozzle 1 so as to be connected to any of the irradiation ports 5A to 5C, and uses the deflecting electromagnets 31 of the beam transport system 30 to guide the particle beam to the irradiation port connected to the irradiation nozzle 1. Thus, the isocenter 8 in which the target volume of the patient 6 lying on the table top 4A of the patient support 4 is present can be irradiated with particle beams from three directions different from one another.

The movement mechanism for moving the irradiation nozzle 1 is not limited to the nozzle rotating system 3. For example, the movement mechanism may be a rotating gantry or the like.

Hereinafter, the disposition of the X-ray imaging apparatus of the radiotherapy apparatus 100 will be described. In the following description, a perpendicular direction is a Z direction, a direction along a rotation axis of the irradiation nozzle 1 in a horizontal direction is a Y direction, and a direction orthogonal to the Y direction in the horizontal direction is an X direction. In this case, the X direction is a direction along a straight line connecting the isocenter 8 and the irradiation port 5A (the irradiation nozzle 1 disposed in the horizontal direction) when viewed from the Z direction. Further, a direction from the isocenter 8 toward the irradiation port 5A is a positive direction in the X direction, and an upward direction in the perpendicular direction is a positive direction in the Z direction. Furthermore, the positive direction in the Y direction is a direction in which the XYZ direction constitutes a right-handed system.

FIG. 3 is views illustrating a disposition example of the X-ray imaging apparatus. Specifically, FIG. 3 (a) schematically illustrates a cross section of an irradiation system 10 taken along a ZX plane passing through the isocenter 8, and FIG. 3 (b) schematically illustrates a cross section of the irradiation system 10 taken along a YZ plane passing through the isocenter 8.

As described above, the X-ray imaging apparatus includes the Xray tube 7 which is the light source unit that irradiates the patient 6 with an X-ray as imaging light, and the FPD2 which is the detection unit that detects the X-ray from the Xray tube 7 through the patient 6. A plurality of combinations of the FPD2 and the Xray tube 7 may be present. In the present embodiment, two combinations of the FPD2 and the Xray tube 7 are present. Specifically, two FPDs 2A and 2B are provided as the FPD2, and two Xray tubes 7A and 7B are provided as the Xray tube 7.

The FPD2A is provided at a position facing the Xray tube 7A with the isocenter 8 interposed therebetween, and the FPD2B is provided at a position facing the Xray tube 7B with the isocenter 8 interposed therebetween. Thus, the FPD2A detects an X-ray emitted from the Xray tube 7A through the patient 6, and detects an X-ray emitted from the Xray tube 7B through the patient 6. The X-ray imaging apparatus is disposed such that a line connecting the FPD2A and the Xray tube 7A and a line connecting the FPD2B and the Xray tube 7B, i.e., a traveling direction of the X-ray from the Xray tube 7A and a traveling direction of the X-ray from the Xray tube 7B intersect.

The X-ray imaging apparatus including the FPD2 and the Xray tube 7 is disposed at a position offset from a line connecting the irradiation nozzle 1 and the isocenter 8 with respect to each of the irradiation start positions of the irradiation nozzle 1. In the present embodiment, the irradiation nozzle 1 is disposed at a position offset from a motion plane M on which the irradiation nozzle 1 moves. In the present embodiment, the motion plane M is a plane along the ZX plane.

The X-ray imaging apparatus is disposed such that the FPD2 is provided on the upstream of the isocenter 8 in the trajectory of the particle beam with respect to each of the irradiation start positions of the irradiation nozzle 1. Specifically, the upstream of the isocenter 8 refers to a location on the reference plane S which is a plane passing through the isocenter 8 and orthogonal to a line 1 connecting the isocenter 8 and the irradiation nozzle 1, or a side closer to the irradiation nozzle 1 than the reference plane S. FIG. 3 (a) illustrates the line 1 and the reference plane S when the irradiation nozzle 1 is oriented in an approximately horizontal direction.

In the example of FIG. 3, the FPDs 2A and 2B and the Xray tubes 7A and 7B are disposed on the reference plane S when the irradiation nozzle 1 is oriented in the approximately horizontal direction. The Xray tubes 7A and 7B are provided at positions lower than the isocenter 8, and the FPDs 2A and 2B are provided at positions higher than the isocenter 8. The X-ray imaging apparatus is disposed such that a line connecting the FPD2A and the Xray tube 7A and a line connecting the FPD2B and the Xray tube 7B are approximately orthogonal to each other.

FIG. 4 is views illustrating another disposition example of the X-ray imaging apparatus in the radiotherapy apparatus 100.

Also in the example of FIG. 4, similarly to the example of FIG. 3, the X-ray imaging apparatus is disposed at a position offset from a line connecting the irradiation nozzle 1 and the isocenter 8 with respect to each of the irradiation start positions of the irradiation nozzle 1, and the FPD2 is provided on the upstream of the isocenter 8 in the trajectory of the particle beam.

However, in the example of FIG. 4, unlike the example of FIG. 3, the X-ray imaging apparatus is disposed on the reference plane S when the irradiation nozzle 1 is oriented in an approximately vertical direction. In other words, all of the FPD2 and the Xray tube 7 are disposed on a plane having the same height as the isocenter 8.

The X-ray imaging apparatus may be disposed on the reference plane S in a case where the irradiation nozzle 1 is oriented in an oblique direction.

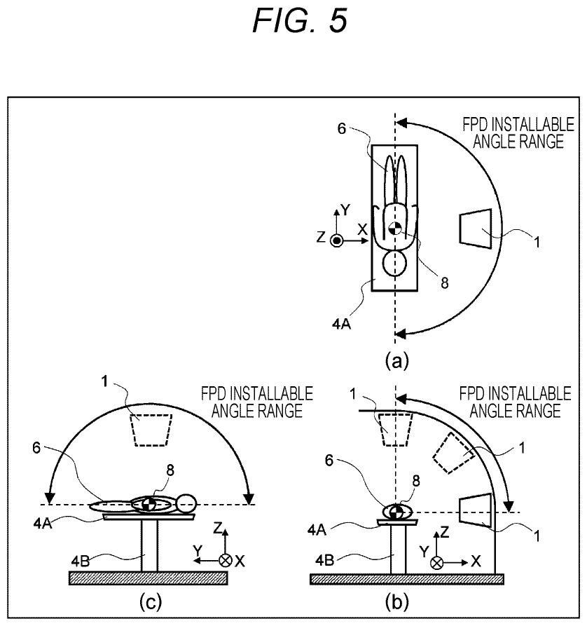

FIG. 5 is views illustrating an FPD installable angle range: a range in which the FPD2 can be disposed in the present embodiment. Specifically, FIG. 5 (a) illustrates the FPD installable angle range on the XY plane, FIG. 5 (b) illustrates the FPD installable angle range on the XZ plane, and FIG. 5 (c) illustrates the FPD installable angle range on the YZ plane. Note that the FPD installable angle range is represented by, for example, a deflection angle range on each of the planes.

In the XY plane direction, as illustrated in FIG. 5 (a), the FPD installable angle range is a deflection angle range from −90° to 90° (i.e., a range in which the X direction is positive). In the XZ plane direction, as illustrated in FIG. 5 (b), the FPD installable angle range is a range where the deflection angle is from 0° to 90° (i.e., a range in which the X direction is positive and the Z direction is positive). In the YZ plane direction, as illustrated in FIG. 5 (c), the FPD installable angle range is a range where the deflection angle is from 0° to 1800° (i.e., a range in which the Z direction is positive).

The radiotherapy apparatus 100 described above may irradiate the patient 6 with an X-ray from the Xray tube 7 of the X-ray imaging apparatus while the irradiation nozzle 1 emits radiation in order to perform a tumor tracking irradiation process of accurately irradiating the organ moving in response to the respiration of the patient with the radiation.

As described above, according to the present embodiment, the X-ray imaging apparatus includes the Xray tube 7 that irradiates the patient 6 with an X-ray as light for imaging, and the FPD2 that detects the X-ray through the patient 6. The Xray tube 7 and the FPD 2 are disposed at positions offset from a line connecting the irradiation nozzle 1 and the isocenter 8 of radiation from the irradiation nozzle 1. The FPD 2 is disposed on a reference plane S which is a plane passing through the isocenter 8 and orthogonal to the line connecting the irradiation nozzle 1 and the isocenter 8, or on a side closer to the irradiation nozzle than the reference plane S. Therefore, it is possible to prevent the secondary particle beam scattered by the radiation from being detected by the FPD2, and thus it is possible to suppress an influence of radiation on the X-ray imaging apparatus.

Further, in the present embodiment, the nozzle rotating system 3 moves the irradiation nozzle 1 and installs the irradiation nozzle 1 at any one of the plurality of irradiation start positions. The FPD2 is disposed on the reference plane S or a side closer to the irradiation nozzle portion than the reference plane S with respect to all of the irradiation start positions. Accordingly, even when radiation is emitted from any irradiation start position, it is possible to prevent the X-ray interfering with the radiation from being detected by the FPD2. Therefore, even when the irradiation nozzle 1 is moved, it is possible to suppress an influence of radiation on the X-ray imaging apparatus.

In the present embodiment, the X-ray imaging apparatus is disposed such that a line connecting the FPD2A and the Xray tube 7A and a line connecting the FPD2B and the Xray tube 7B are approximately orthogonal to each other. Further, the FPDs 2A and 2B and the Xray tubes 7A and 7B are disposed on the reference plane S in a case where the irradiation nozzle 1 is installed at any one of the irradiation start positions. In this case, it is possible to more appropriately suppress the influence of radiation on the X-ray imaging apparatus.

In the present embodiment, the Xray tube of the X-ray imaging apparatus irradiates the patient 6 with an x-ray while the irradiation nozzle 1 emits radiation. Therefore, it is possible to prevent the secondary particle beam scattered by the radiation from being detected by the FPD2, and thus, it is possible to accurately perform the tumor tracking irradiation process and the like.

In the present embodiment, the radiation is a carbon ion beam. Since the carbon ion beam has a larger influence on the X-ray than other radiation, the effect of suppressing the influence on the X-ray imaging apparatus is also increased.

### Second Embodiment

In the first embodiment, the irradiation nozzle 1 rotates by 90°, but in the present embodiment, the irradiation nozzle 1 is fixed. The X-ray imaging apparatus is disposed at a position offset from a line connecting the irradiation nozzle 1 and the isocenter 8 with respect to the fixed irradiation nozzle 1, and the FPD2 is provided on the upstream of the isocenter 8 in the trajectory of the particle beam.

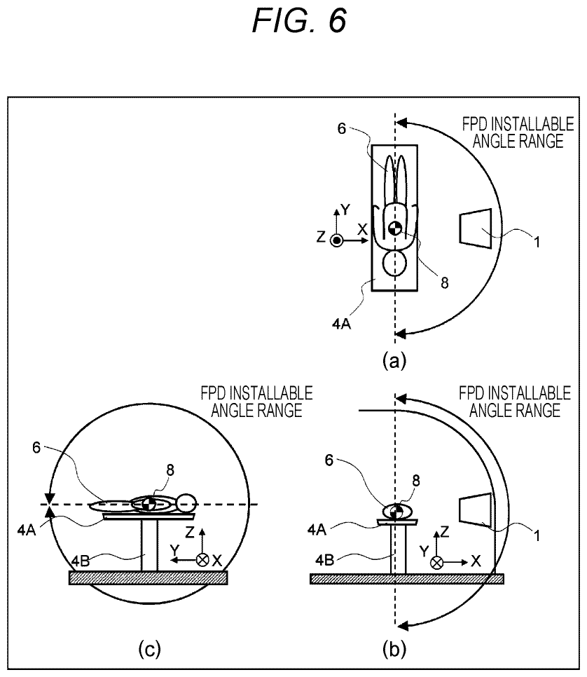

FIG. 6 is views illustrating an FPD installable angle range of the FPD2 in a case where the irradiation nozzle 1 is fixed. Specifically, FIG. 6 (a) illustrates the FPD installable angle range on the XY plane, FIG. 6 (b) illustrates the FPD installable angle range on the XZ plane, and FIG. 6 (c) illustrates the FPD installable angle range on the YZ plane. In the example of FIG. 6, the irradiation nozzle 1 is fixed in the horizontal direction.

In the XY plane direction, as illustrated in FIG. 6 (a), the FPD installable angle range is a deflection angle range from −90° to 90° (i.e., a range in which the X direction is positive). In the XZ plane direction, as illustrated in FIG. 6 (b), the FPD installable angle range is a range where the deflection angle is from −90° to 90° (i.e., a range in which the X direction is positive). In the YZ plane direction, as illustrated in FIG. 5 (c), the FPD installable angle range is a range where the deflection angle is from 0° to 360° (i.e., the entire range in the Z direction).

Also in the present embodiment, it is possible to prevent the secondary particle beam scattered by the radiation from being detected by the FPD2, and thus it is possible to suppress an influence of radiation on the X-ray imaging apparatus.

### Third Embodiment

In the present embodiment, an example in which a plurality of fixed irradiation nozzles 1 is present will be described.

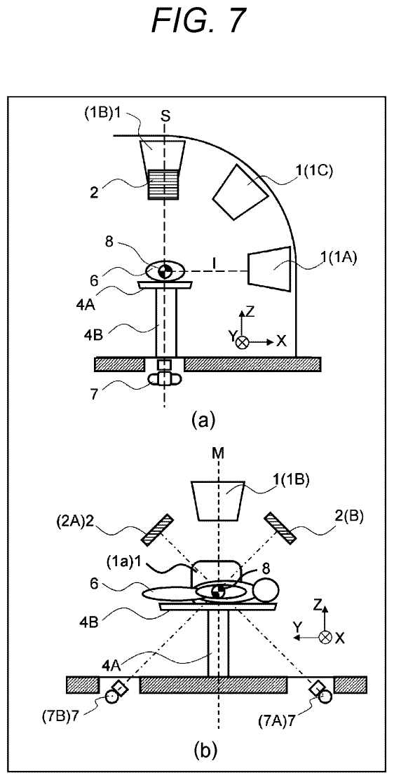

FIG. 7 is views illustrating a disposition example of the X-ray imaging apparatus. Specifically, FIG. 7 (a) schematically illustrates a cross section along the ZX plane passing through the isocenter 8 of the patient 6 of the irradiation system 10, and FIG. 7 (b) schematically illustrates a cross section along the YZ plane passing through the isocenter 8 of the patient 6 of the irradiation system 10.

The irradiation system 10 illustrated in FIG. 7 is different from the irradiation system 10 illustrated in FIG. 2 in that a plurality of irradiation nozzles 1 is included. Specifically, in the example of FIG. 7, two irradiation nozzles 1A and 1B are provided as the irradiation nozzle 1.

The irradiation nozzles 1A to 1C are provided at the same positions as the plurality of irradiation start positions in the first embodiment. That is, the irradiation nozzle 1A is connected to the irradiation port 5A in FIG. 2, the irradiation nozzle 1B is connected to the irradiation port 5B in FIG. 2, and the irradiation nozzle 1C is connected to the irradiation port 5C in FIG. 2.

The X-ray imaging apparatus is disposed at a position offset from a line connecting the irradiation nozzle 1 and the isocenter 8 with respect to each of the irradiation nozzles 1, and the FPD2 is provided on the upstream of the isocenter 8 in the trajectory of the particle beam. In this case, the FPD installable angle range in which the FPD2 can be disposed is the same as the FPD installable angle range of the first embodiment illustrated in FIG. 5.

The number of the irradiation nozzles 1 is not particularly limited, and for example, two irradiation nozzles may be used.

Also in the present embodiment, it is possible to prevent the secondary particle beam scattered by the radiation from being detected by the FPD2, and thus it is possible to suppress an influence of radiation on the X-ray imaging apparatus.

### Fourth Embodiment

In the first embodiment, the irradiation nozzle 1 rotates by 90°, but the present embodiment, the irradiation nozzle 1 rotates by 180°.

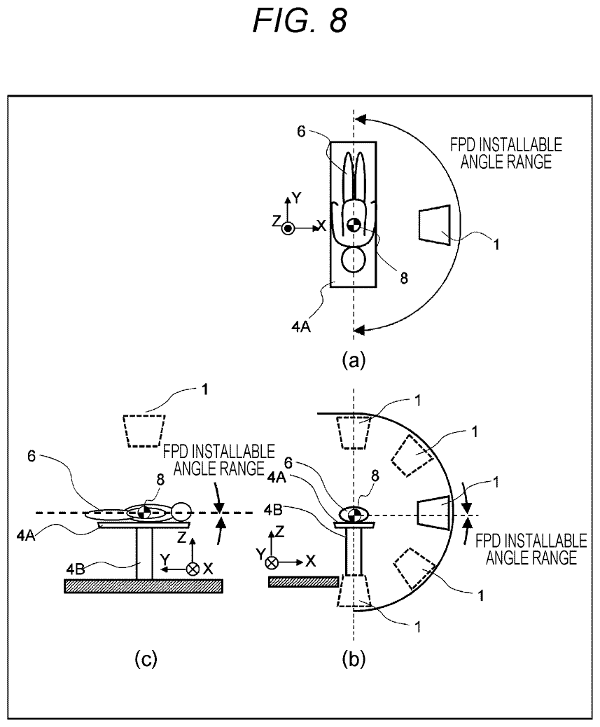

FIG. 8 is a view illustrating an FPD installable angle range of the FPD2 in a case where the irradiation nozzle 1 rotates by 180°. Specifically, FIG. 8 (a) illustrates the FPD installable angle range on the XY plane, FIG. 8 (b) illustrates the FPD installable angle range on the XZ plane, and FIG. 8 (c) illustrates the FPD installable angle range on the YZ plane.

In the example of FIG. 8, the irradiation nozzle 1 rotates 180° from upward in the vertical direction (positive direction in the Z direction) to downward in the vertical direction (negative direction in the Z direction). Although not illustrated in FIG. 7, five irradiation ports 5 are present, and the irradiation ports 5 are oriented downward in the vertical direction, a direction at an angle of approximately 45 degrees with respect to the horizontal direction, a direction at an angle of about −45 degrees with respect to the horizontal direction, and upward in the vertical direction, respectively. The irradiation nozzle 1 is moved by the nozzle rotating system 3 so as to be connected to any one of the five irradiation ports 5.

In the case of FIG. 8, in the XY plane direction, as illustrated in FIG. 8 (a), the FPD installable angle range is a deflection angle range from −90° to 90°(i.e., a range in which the X direction is positive). In the XZ plane direction, as illustrated in FIG. 8 (b), the FPD installable angle range is a value at only the position where the deflection angle is 0°. In the YZ plane direction, as illustrated in FIG. 8 (c), the FPD installable angle range is a value at only the position where the deflection angle is 0°.

Also in the present embodiment, it is possible to prevent the secondary particle beam scattered by the radiation from being detected by the FPD2, and thus it is possible to suppress an influence of radiation on the X-ray imaging apparatus.

The above-described embodiments of the present disclosure are examples for describing the present disclosure, and are not intended to limit the scope of the present disclosure only to those embodiments. Those skilled in the art can practice the present disclosure in various other aspects without departing from the scope of the present disclosure.

### REFERENCE SIGNS LIST

1, 1A, 1B, 1C irradiation nozzle
2, 2A, 2B FPD
3 nozzle rotating system
4 patient support
4A table top
4B arm part
5, 5A, 5B, 5C irradiation port
6 patient
7, 7A, 7B Xray tube
8 isocenter
10 irradiation system
20 accelerator
21 linear accelerator
22 synchrotron
30 beam transport system
31, 31A bending magnet
100 radiotherapy apparatus

1. A radiotherapy apparatus comprising:
an irradiation unit which irradiates an irradiation object with radiation; and
an imaging apparatus which takes an image of the irradiation object,
the imaging apparatus comprising:
a light source unit that irradiates the irradiation object with light for imaging; and a detection unit that detects the light through the irradiation object,
wherein the light source unit and the detection unit are disposed at positions offset from a line connecting an isocenter of the radiation and the irradiation unit, and
the detection unit is disposed on a reference plane which is a plane passing through the isocenter and orthogonal to the line connecting the isocenter and the irradiation unit or on a side closer to the irradiation unit than the reference plane.

2. The radiotherapy apparatus according to claim 1, further comprising a movement mechanism which moves the irradiation unit to be installed at any one of a plurality of irradiation start positions, wherein the detection unit is disposed on the reference plane or on the side closer to the irradiation unit than the reference plane, with respect to all of the plurality of irradiation start positions.

3. The radiotherapy apparatus according to claim 1, wherein the irradiation unit is plural, and
the detection unit is disposed on the reference plane or on the side closer to the irradiation unit than the reference plane, with respect to each of the plurality of irradiation units.

4. The radiotherapy apparatus according to claim 1, wherein the imaging apparatus includes two combinations of the light source unit and the detection unit, and
two lines connecting the light source unit and the detection unit in each of the two combinations are approximately orthogonal to each other, and the imaging apparatus is disposed on the reference plane.

5. The radiotherapy apparatus according to claim 1, wherein the light source unit emits the light while the irradiation unit emits the radiation.

6. The radiotherapy apparatus according to claim 1, wherein the radiation is a carbon ion beam.
